(19) United States
(12) Patent Application Publication
(10) Pub. No.: US 2017/0028091 A1
(43) Pub. Date: Feb. 2, 2017

(54) SCENT DIFFUSER, METHOD FOR MAKING A SCENT DIFFUSER AND METHOD FOR DIFFUSING A SCENT

(71) Applicants: Luc DESGAGNE, St-Basile-le-Grand (CA); Héléne GALARNEAU, Mirabel (CA)

(72) Inventors: Luc DESGAGNE, St-Basile-le-Grand (CA); Héléne GALARNEAU, Mirabel (CA)

(21) Appl. No.: 14/809,677

(22) Filed: Jul. 27, 2015

(51) Int. Cl. A61L 9/12; B65D 81/26

(52) U.S. Cl. CPC A61L 9/127; B65D 81/266

(57) ABSTRACT

A scent diffuser comprises a porous membrane, an absorbing material and a scented liquid. The absorbing material is substantially contained by the porous membrane. The absorbing material contains peat moss. The scented liquid is absorbed by the absorbing material. When the scent diffuser is in use, the absorbing material gradually releases the absorbed scented liquid in ambient air through the porous membrane so as to distribute over a period of time a scent of the scented liquid.

### FIELD OF THE INVENTION

The present invention generally relates to the field of scent diffusers. More specifically, the invention relates to a scent diffuser that uses peat moss as an absorbing material to absorb and release a scented liquid.

### BACKGROUND OF THE INVENTION

Diffusing a nice scent to mask an unpleasant smell such as those that may occur in bathrooms is common practice. Aromatherapy, which is a method of diffusing soothing or stimulating scents, depending on an intended objective, is rapidly gaining in popularity. Olfactive marketing consists of using scents in a business context to enhance the experience or to alter the mood of customers and to eventually make the customer take a purchasing decision. What all these activities have in common is that they use some kind of scent diffuser to diffuse the scent in ambient air.

Some diffuser atomize a scented liquid directly in the air to produce the intended scent. The drawback of such scent diffusers is that the scent typically does not last long. Hence, a rather large quantity of scented liquid is required over time.

In order to make the scent last longer, some diffusers use an absorbent material which absorbs the scented liquid and slowly release it in the ambient air. Such scent diffusers use a wick as its absorbent materials. The wick may be made of a rope, paper, or a bamboo stick. They all work by capillarity. Although they make the smell last longer than a spray, they often waste much scented liquid in the air through direct evaporation of the scented liquid in which the wick sit. This produces a more present smell in the first few hours or days, which rapidly declines as the scented liquid evaporates relatively rapidly. Other types of absorbent materials may include a gel or a sponge, which often are not natural products.

Some other diffuser use energy to heat the scented liquid in order to release it in gas form in the ambient air. The disadvantage of such technology is that it requires a source of energy, which may not always be conveniently located or at all present in the room where the scent is intended to be released.

In terms of scented liquids, many are petrol derived products, which may have a negative long-term incidence on health as the invisible particles of scented liquid suspended in ambient air are breathed and then absorbed by humans.

There is therefore a need for an air diffuser that does not use energy, that do not use petrol by-products and that provides a longer-lasting scent.

### SUMMARY OF THE INVENTION

It is an object of the present invention to provide a scent diffuser that overcomes or mitigates one or more disadvantages of known scent diffusers, or at least provides a useful alternative.

In accordance with an embodiment of the present invention, there is provided a scent diffuser for releasing a scent in ambient air. The scent diffuser comprises a porous membrane, an absorbing material and a scented liquid. The absorbing material, which absorbs the scented liquid, is substantially contained by the porous membrane. The absorbing material contains peat moss. When the scent diffuser is in use, the absorbing material gradually releases the scented liquid in the air through the porous membrane so as to distribute over a period of time a scent of the scented liquid that was absorbed by the absorbing material.

The porous membrane may be opaque and made of a fabric. The fabric may be a synthetic fiber such as nylon or a natural fiber such as jute or cotton for example.

The scented liquid may be a scented oil such as an essential oil made of natural products.

The absorbing material is substantially made of peat moss. Optionally, the absorbing material may be completely made of peat moss. The peat moss may be made of dried particles having a diameter smaller than 6 mm (0.24 inch) and preferably smaller than 5 mm (0.20 inch).

Optionally, the scent diffuser may further include a container in which the absorbing material and the scented liquid are contained. The absorbing material and the scented liquid absorbed by the absorbing material may be placed directly in the container, or may be completely contained within the porous membrane. The container has an aperture through which the scent of the scented liquid is released. The aperture is at least partially closed by the porous membrane if the absorbing material with the scented liquid is placed completely within a closed bag made of the porous material, or the aperture may be completely closed by the porous membrane if the absorbing material with the scented liquid is placed directly in the container.

The container is preferably opaque and may be made of a metallic material such as aluminum.

In accordance with another embodiment of the present invention, there is provided a scent diffuser for diffusing a scent in ambient air where the scent diffuser comprises a porous membrane, an absorbing material, a scented liquid and a container. The absorbing material is substantially contained by the membrane and is made with peat moss. Optionally, the absorbing material may be made completely of peat moss. The scented liquid is absorbed by the absorbing material. The absorbing material with the absorbed scented liquid are contained within the container. The container has an aperture to release the scent of the scented liquid. The aperture is at least partially closed by the porous membrane. When the scent diffuser is in use, the absorbing material gradually releases the scented liquid in the ambient air through the porous membrane so as to distribute over a period of time a scent of the scented liquid that was absorbed by the absorbing material.

Advantageously, the absorbing material and the absorbed scented liquid may be completely contained by the porous material such as if placed in a bag. Alternatively, the absorbing material and the absorbed scented liquid may be directly placed in the container and the porous membrane be placed so as to close the aperture, thereby preventing the absorbing material to escape the container while still allowing the scent particles of the scented liquid to escape in the ambient air.

Preferably, the container is made of aluminum and the scented liquid is an essential oil.

In accordance with yet another embodiment of the present invention, there is provided a method for making a scent diffuser. The method comprises the step of containing with a porous membrane an absorbing material made of peat moss and a scented liquid absorbed by the absorbing material.

Optionally, the method further comprises placing the porous membrane in a container.

Preferably, the method further comprises substantially closing an aperture of the container with the porous membrane.

In accordance with still another embodiment of the present invention, there is provided a method of diffusing a scent. The method comprises gradually releasing through a porous membrane a scented liquid absorbed by an absorbing material where the absorbing material is substantially made of peat moss.

### BRIEF DESCRIPTION OF DRAWINGS

These and other features of the present invention will become more apparent from the following description in which reference is made to the appended drawings wherein:

FIG. 1 is an isometric view of a scent diffuser shown partially open to expose the material contained inside the scent diffuser in accordance with an embodiment of the present invention;

FIG. 2 is an isometric view of the scent diffuser of FIG. 1 placed in a container and shown partially open to expose the material contained inside the scent diffuser;

FIG. 3 is a schematic view describing a method of making a scent

### DETAILED DESCRIPTION OF THE INVENTION

The present invention relates to a scent diffuser. The scent diffuser uses a natural absorbing material which slowly and gradually releases a scented liquid absorbed by an absorbing material substantially made of peat moss through a porous membrane. The absorbing material may be reused.

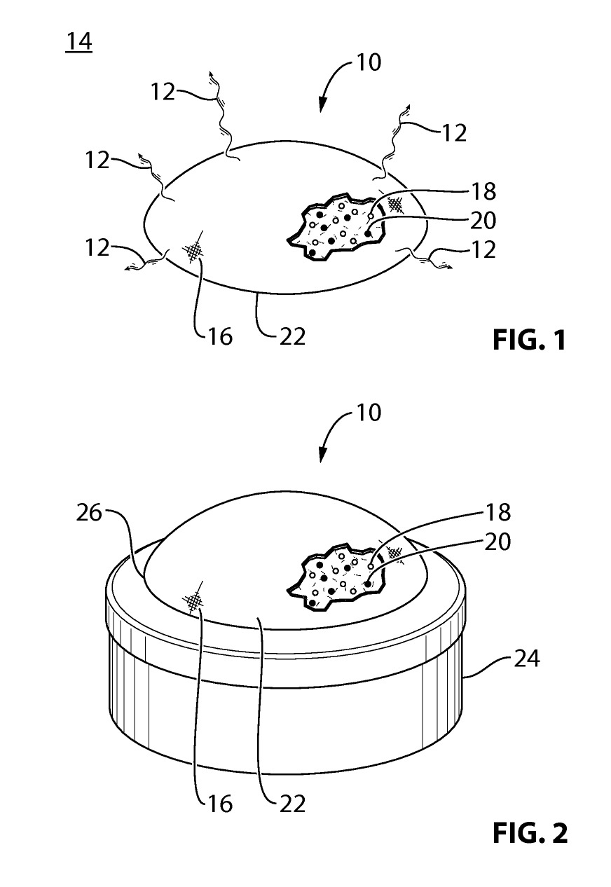

FIG. 1 depicts a scent diffuser 10 for diffusing a scent 12 in ambient air 14. The scent diffuser 10 comprises a porous membrane 16, an absorbing material 18 and a scented liquid 20 absorbed by the absorbing material 14.

The scented liquid 20 is first mixed with the absorbing material 18 so that the scented liquid is all absorbed by the absorbing material 18. The absorbing material 18, soaked with the scented liquid 20, is then placed inside a bag 22 made of the porous membrane 16. The absorbing material 18 then slowly and gradually releases particles of the scented liquid 20 in the ambient air 14 through the porous membrane 16.

Typically, the scented liquid 20 is made of scented oil. For those who prefer natural products, essential oils having different fragrances may be used. Such oils are commercially available.

Because essential oils may be affected by ultra-violet rays, it is preferable in this case to use a porous membrane 16 that is opaque. The porous membrane 16 is typically made of a fabric, itself made of a synthetic fiber such as nylon, which may be blended with lycra to provide some elasticity, or of a natural fiber such as cotton or jute. The porous membrane material needs to be selected so that the weave of the fabric does not create pores that are so large so as to let the absorbing material 18 through, or too small so as to prevent the released scented particles of the scented liquid 20 to travel through the porous membrane 16. It has been found that widely available fabrics, such as cotton used for T-shirts, performs well.

The absorbing material 18 is made in major part of peat moss. Actually, it has been found that it may be preferable to solely use peat moss as the absorbing material 18. It has been found that a fine-grained peat moss with dried particles having a diameter smaller than 6 mm (0.24 inch), and preferably smaller or equal to 5 mm (0.20 inch) performs very well in retaining the scented liquid 20 and slowly releasing it. For example, the absorbing material “Clean Sorb”, an organic absorbent made of sphagnum peat moss performs very well. This absorbing material is made by Lambert, a Canadian company. This type of absorbing material may absorb up to 13 times its weight in liquid. It has been found that a ratio of 40 ml (2.4 cubic inch) of scented liquid 20 absorbed in 100 ml (6.1 cubic inch) of absorbing material 18 performs adequately.

Once the absorbing material 18 has absorbed the scented liquid 20, by trapping or encapsulating the scented liquid 20, it slowly and gradually releases particles of the scented liquid 20. For the quantities provided above, the scent may last up to a month. Once the absorbing material 18 has released all or most of the scented liquid 20 in the air 14, it may conveniently be re-used to absorb more scented liquid 20, thereby making for a user-friendly and earth-friendly scent diffuser.

For convenience, the scent diffuser 10 may use a container 24 in which the bag 22, containing the absorbing material 18 having absorbed the scented liquid 20, is placed. The container 24 is equipped with an aperture 26 to release the scent of the scented liquid 20. The bag 22 is typically placed in the container 24 so as to completely close the aperture 26. However, it may occur that the bag 22 does not completely close the aperture 26. This does not create a real problem as the absorbing material 18 is totally contained within the bag 22 made of the porous membrane 16.

Alternatively, the absorbing material 18 having absorbed the scented liquid 20 may be placed directly in the container 24. In this case, it is preferable that the porous membrane 16 be placed so as to completely close the aperture 26 so that the absorbing material 18 cannot escape. In this case, the porous membrane 16 may be attached on a perimeter of the aperture 26.

Again, because essential oils, which may be used as the scented liquid 20, are affected by ultra-violets rays, it may be preferable to use an opaque container. The container 24 may be made of dark colored glass, painted glass, wood, plastic, metallic material, or any other suitable material. It has been found that an aluminum container performs adequately, may be cleaned easily and does not get degraded by the scented liquid 20.

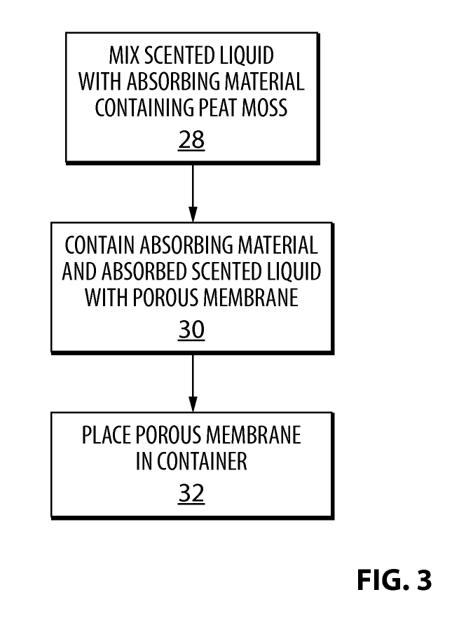

FIG. 3 is now referred to. The scent diffuser 10 may be made by first mixing a first quantity of scented liquid 20 in a second quantity of absorbing material 18 at step 28 until the scented liquid 20 is fully absorbed by the absorbing material 18, or until the absorbing material 18 is saturated. These mixed components are then placed either directly in the bag 22 made of the porous membrane 16, which is then closed to control diffusion of the scented liquid 16, or directly in the container 24 in step 30. The importance being that the mixed components be at least partially contained by the porous membrane 16. In the case where the mixed components is placed in the bag 22, the bag 22 is closed and then may be placed in the container 24 at step 32. In the case where the mixed components of absorbing material 18 and absorbed scented liquid 20 are placed directly in the container 24, the porous membrane 16 must be installed so as to close the aperture of the container 24 and so as to contain these mixed components and allow gradual release of the scented liquid 20.

Different fragrances may be used for different applications of the scent diffuser 10 and its associated method of diffusing a scent. For example, a scented liquid having a coffee fragrance may be used near a coffee machine or near coffee shops to entice customers to purchase coffee. Another example may be to use the scent diffuser 10 and associated method to diffuse a fragrance of lemon in a public bathroom to mask odors. Yet another example may be to use the scent diffuser 10 and associated method to diffuse a fragrance of lavender in a house to provide a soothing atmosphere.

The present invention has been described with regard to preferred embodiments. The description as much as the drawings were intended to help the understanding of the invention, rather than to limit its scope. It will be apparent to one skilled in the art that various modifications may be made to the invention without departing from the scope of the invention as described herein, and such modifications are intended to be covered by the present description. The invention is defined by the claims that follow.

1. A scent diffuser for diffusing a scent in ambient air, the scent diffuser comprising:
a porous membrane;
an absorbing and releasing material, said absorbing and releasing material being substantially contained by said porous membrane, said absorbing and releasing material having peat moss;
a scented liquid, said scented liquid having a scent, said scented liquid being absorbed by said absorbing and releasing material,
wherein when said scent diffuser is in use, said absorbing and releasing material gradually releases said absorbed scented liquid in the ambient air through said porous membrane so as to distribute over a period of time said scent of said scented liquid absorbed by said absorbing and releasing material.

2. The scent diffuser of claim 1 wherein said porous membrane is opaque.

3. The scent diffuser of claim 2 wherein said porous membrane is made of a fabric.

4. The scent diffuser of claim 3 wherein said fabric is made of a natural fiber.

5. The scent diffuser of claim 3 wherein said fabric is made of either one of nylon, jute or cotton.

6. The scent diffuser of claim 1 wherein said scented liquid is a scented oil.

7. The scent diffuser of claim 6 wherein said scented oil is an essential oil made from natural products.

8. The scent diffuser of claim 1 wherein said absorbing material is substantially made of said peat moss.

9. The scent diffuser of claim 8 wherein said peat moss is made of dried particles having a diameter smaller than 6 mm (0.24 inch).

10. The scent diffuser of claim 1 further comprises a container, said absorbing and releasing material and said absorbed scented liquid being contained within said container, said container having an aperture to release the scent of said scented liquid, said aperture being at least partially closed by said porous membrane.

11. The scent diffuser of claim 10 wherein said absorbing and releasing material is completely contained by said porous membrane.

12. The scent diffuser of claim 10 wherein said aperture is totally closed by said porous membrane.

13. The scent diffuser of claim 10 wherein said container is opaque.

14. The scent diffuser of claim 13 wherein said container is made of aluminum.

15. A scent diffuser for diffusing a scent in ambient air, the scent diffuser comprising:
a porous membrane;
an absorbing and releasing material, said absorbing and releasing material being substantially contained by said porous membrane, said absorbing and releasing material having peat moss;
a scented liquid, said scented liquid having a scent, said scented liquid being absorbed by said absorbing and releasing material;
a container, said absorbing and releasing material and said absorbed scented liquid being contained within said container, said container having an aperture to release a scent of said scented liquid, said aperture being at least partially closed by said porous membrane,
wherein when said scent diffuser is in use, said absorbing and releasing material gradually releases particles of said scented liquid in the air through said porous membrane so as to distribute over a period of time said scent of said scented liquid absorbed by said absorbing and releasing material.

16. The scent diffuser of claim 15 wherein said absorbing and releasing material is completely contained by said porous membrane, said absorbing and releasing material is substantially made of said peat moss, said container is made of aluminum and said scented liquid is an essential oil.

17. A method for making a scent diffuser comprising:
containing in a porous membrane an absorbing material made of peat moss and a scented liquid absorbed by said absorbing material.

18. The method of claim 17 further comprising placing said porous membrane in a container.

19. The method of claim 19 further comprising substantially closing an aperture of said container with said porous membrane.

20. A method of diffusing a scent comprising gradually releasing through a porous membrane a scented liquid absorbed by an absorbing material substantially made of peat moss.
